(19) United States
(12) Patent Application Publication
(10) Pub. No.: US 2025/0222076 A1
(43) Pub. Date: Jul. 10, 2025

(54) USE OF p55gamma PROTEIN OR ENCODING GENE THEREOF AS TARGET IN PREPARATION OF DRUG FOR PREVENTING AND/OR TREATING VASCULAR CALCIFICATION

(71) Applicant: Beijing Friendship Hospital, Capital Medical University, Beijing (CN)

(72) Inventors: Chunmei CAO, Beijing (CN); Weiwei AN, Beijing (CN); Yiyang JIANG, Beijing (CN); Yang LI, Beijing (CN); Yingjiao JU, Beijing (CN)

(73) Assignee: Beijing Friendship Hospital, Capital Medical University, Beijing (CN)

(21) Appl. No.: 18/764,595

(22) Filed: Jul. 5, 2024

(30) Foreign Application Priority Data
Jan. 9, 2024 (CN) 202410032170.1

(51) Int. Cl. A61K 38/45; A61K 48/00; A61P 9/10; C12N 15/86

(52) U.S. Cl. CPC A61K 38/45; A61K 48/0033; A61P 9/10; C12N 15/86; C12N 2710/10043; C12N 2710/10051

(57) ABSTRACT

Use of a p55gamma protein or an encoding gene thereof as a target in the preparation of a drug for preventing and/or treating vascular calcification is provided, belonging to the technical field of vascular calcification prevention and treatment. The use of the p55gamma protein or the encoding gene thereof as the target in the preparation of the drug for preventing and/or treating the vascular calcification is proposed for the first time, and the p55gamma protein affects the vascular calcification by regulating phosphorylation of Yes-associated protein (YAP). It is proposed for the first time that the vascular calcification can be prevented and/or treated by overexpressing p55gamma gene or increasing the activity of the p55gamma protein.

### CROSS REFERENCE TO RELATED APPLICATION

This patent application claims the benefit and priority of Chinese Patent Application No. 202410032170.1 filed with the China National Intellectual Property Administration on Jan. 9, 2024, the disclosure of which is incorporated by reference herein in its entirety as part of the present application.

### REFERENCE TO SEQUENCE LISTING

A computer readable XML file entitled “Sequence Listing”, that was created on Jun. 18, 2024, with a file size of 7,496 bytes, contains the sequence listing for this application, has been filed with this application, and is hereby incorporated by reference in its entirety.

### TECHNICAL FIELD

The present disclosure belongs to the technical field of vascular calcification prevention and treatment, and particularly relates to use of p55gamma protein or its encoding gene as a target in the preparation of a drug for preventing and/or treating vascular calcification.

### BACKGROUND

Atherosclerosis is a common pathophysiological process in various cardiovascular diseases. During the formation of atherosclerosis, adaptive intimal hyperplasia and foam cells infiltration occur at the lesion site, and then lipid lakes are formed, vascular smooth muscle cells are involved in the formation of fibrous caps, and finally calcification occurs. Vascular calcification, an important component of the atherosclerosis, refers to the pathological process that calcium and phosphate are excessively deposited on blood vessel walls. Clinically, calcifications that can detected by computed tomograph (CT), generally larger than 5 μm in diameter, are referred to as “macrocalcifications”; while those that are smaller than 5 μm and cannot be detected by CT are referred to as “microcalcifications”. The current mainstream view is that vascular calcification is an active and multi-factor-regulated process similar to bone mineralization, which includes osteoblast-like cell differentiation, matrix maturation, and mineralization. The vascular calcification involves complex mechanisms, including oxidative stress, autophagy, apoptosis, endoplasmic reticulum stress, imbalance between calcification promoters and inhibitors, hormonal regulation, matrix remodeling, and matrix vesicle formation. However, there is still a lack of effective treatment strategies for the vascular calcification, and the endogenous inhibitors of vascular calcification still remains unclear.

The pathophysiological process of vascular smooth muscle cell calcification typically begins in the necrotic core of atherosclerosis. The initiation of calcification generally originates from macrophages and vascular smooth muscle cells that undergo phenotypic transformation of osteoblast-like cells. These cells release substances such as matrix vesicles, exosomes, and apoptotic bodies to provide deposition and attachment sites for the initiation of calcification. The concentrations of calcium ions and phosphates gradually increase as the deposition and attachment site gradually loses regulation of calcium and phosphate concentrations. In the presence of inorganic pyrophosphate, alkaline phosphatase and ATPase decompose macromolecule of the inorganic pyrophosphate to produce free phosphates. The vascular calcification is also regulated by apoptosis. Proudfoot et al. found that stimulating human vascular smooth muscle cell apoptosis with the combination of anti-Fas-IgM and cyclohexylamine can increase the formation of vascular calcification; while inhibiting apoptosis with caspase inhibitors can reduce the occurrence of vascular calcification. Studies have also found that the DNA fragmentation characteristics of apoptosis were observed in vascular smooth muscle cells on the 7th day of culture, but the deposition of calcium crystals is not found until the 28th day. This result indicates that apoptosis precedes calcification, thus further demonstrating the importance of apoptosis in the initiation of vascular calcification. Protein folding and maturation are fundamental processes in the endoplasmic reticulum. The endoplasmic reticulum stress response is triggered once the demands for intracellular protein folding exceed the processing capacity of the endoplasmic reticulum. This stress response leads to the upregulation of molecular chaperones such as the 78-kDa glucose-regulated proteins GRP78 and GRP94, which help stabilize the protein folding. In the rat vascular calcification model, both GRP78 and GRP94 are significantly increased in aortic tissues induced by vitamin D₃, indicating that endoplasmic reticulum stress is involved in the vascular calcification. In addition, the endoplasmic reticulum stress during vascular calcification also activates activating transcription factor 4 (ATF4), which is another important molecule associated with the endoplasmic reticulum stress. In addition to GRP78, GRP94, and ATF4, many other molecules are also involved in the occurrence and development of endoplasmic reticulum stress and vascular calcification. For example, X-box binding protein 1 (XBP1), one of the major regulators involved in endoplasmic reticulum stress, is also involved in the calcification and extracellular matrix remodeling of vascular smooth muscle cells. Additionally, other members of the endoplasmic reticulum stress signaling pathway, such as IRE1 and CHOP, have also been found to be closely related to the occurrence and development of vascular calcification. MicroRNAs (miRNAs), a class of short non-coding RNA, mainly inhibit the translation of mRNA and degrade the mRNA by binding to the 3′-untranslated region of the mRNA, thus affecting the regulation of gene expression. In recent years, more and more studies have proved that miRNAs also play an important role in vascular calcification. In 2011, Goettsch et al. first discovered the role of miRNAs in vascular calcification. Furthermore, miRNA-125b was found to promote the transdifferentiation of vascular smooth muscle cells into osteoblast-like cells by regulating the transcription factor Osterix. Later studies have showed that miRNA-141 can promote arterial valve calcification through the bone morphogenetic protein 2 (BMP2) signal transduction pathway. In addition to miR-125b and miRNA-141, many other miRNAs are involved in the occurrence and development of vascular calcification. These miRNAs regulate a variety of cell signaling pathways and molecular mechanisms, including regulating osteoblast differentiation, apoptosis, inflammatory response, matrix remodeling, and extracellular matrix deposition. For example, miR-204 can directly bind to BMP2 mRNA and inhibit the differentiation and mineralization of bone marrow mesenchymal stem cells (BMSCs) by regulating the BMP2/Runx2/ALPL signaling pathway. In the process of aortic valve calcification in rats, miR-29b is involved in the vascular and valvular calcification, and inhibiting miR-29b can reduce calcification and induce TGF-β3 expression. More and more studies have shown that different miRNAs can interact and regulate with each other, forming a complex regulatory network. The aggregation and activation of macrophages and T lymphocytes in diseases such as atherosclerosis and valvular heart disease are also important factors in promoting vascular calcification. Inflammatory mediators and cytokines produced by macrophages and T lymphocytes, such as tumor necrosis factor alpha (TNF-α), IL-6, and IFN-γ, can stimulate the differentiation and matrix deposition of vascular smooth muscle cells into osteoblast-like cells, thereby promoting the progression of vascular calcification. In addition, studies have found that monocytes/macrophages can also enhance the occurrence and development of vascular calcification through cell-cell interactions and the production of soluble factors. High phosphorus also causes extracellular matrix remodeling. The production of matrix metalloproteinases and cysteine proteases leads to matrix protein degradation while promoting collagen synthesis, thereby producing a collagen-rich extracellular matrix to further promote vascular calcification. In summary, vascular calcification is a complex pathophysiological process regulated by multiple mechanisms and genes. As a result, it is of great significance to further explore the molecular mechanisms of vascular calcification.

p55gamma (p55γ) is a phosphatidylinositol-3-kinase (PI3K) regulatory subunit discovered in recent years. Although this regulatory subunit is expressed in the heart and blood vessels, its correlation with vascular calcification has not been reported yet.

### SUMMARY

In view of this, the objective of the present disclosure is to provide use of a p55gamma protein or an encoding gene thereof as a target in the preparation of a drug for preventing and/or treating vascular calcification.

To achieve the above objective, the present disclosure provides the following technical solutions:

The present disclosure provides use of a p55gamma protein or an encoding gene thereof as a target in the preparation of a drug for preventing and/or treating vascular calcification.

In some embodiments, the p55gamma protein affects the vascular calcification by regulating phosphorylation of a Yes-associated protein (YAP).

In some embodiments, this effect is achieved by overexpressing p55gamma gene or increasing an activity of the p55gamma protein.

In some embodiments, a process of overexpressing p55gamma gene includes adenovirus transfection; and the adenovirus transfection specifically includes the following steps: transforming a p55gamma-overexpressed plasmid pAd-track-p55gamma into an Escherichia coli strain BJ5183 to allow recombination, selecting resulting single clones to allow identification by restriction analysis, transforming a resulting successfully-identified single clone into an Escherichia coli strain DH5A to allow bacterial solution amplification and plasmid extraction, and linearizing a resulting plasmid with a restriction endonuclease PAC1; transfecting a resulting linearized plasmid into a 293A cell to produce a recombinant adenovirus particle, and then infecting cells with the recombinant adenovirus particle or injecting the recombinant adenovirus particle into body to achieve the process of overexpressing p55gamma gene.

The present disclosure further provides a method for studying a function of genes related to vascular calcification, or screening a drug for preventing or treating the vascular calcification, or constructing a disease model of the vascular calcification, or analyzing a gene therapy drug of the vascular calcification, including overexpressing p55gamma gene or increasing an activity of the p55gamma protein.

In some embodiments, the method is used to prevent or treat the vascular calcification in a cell in vitro, in an animal model in vivo, or in drug screening.

In some embodiments, a process of overexpressing p55gamma gene includes adenovirus transfection; and the adenovirus transfection specifically includes the following steps: transforming a p55gamma-overexpressed plasmid pAd-track-p55gamma into an Escherichia coli strain BJ5183 to allow recombination, selecting resulting single clones to allow identification by restriction analysis, transforming a resulting successfully-identified single clone into an Escherichia coli strain DH5A to allow bacterial solution amplification and plasmid extraction, and linearizing a resulting plasmid with a restriction endonuclease PAC1; transfecting a resulting linearized plasmid into a 293A cell to produce a recombinant adenovirus particle, and then infecting cells with the recombinant adenovirus particle or injecting the recombinant adenovirus particle into body to achieve the process of overexpressing p55gamma gene.

The present disclosure further provides a drug for preventing and/or treating vascular calcification, where the drug is capable of increasing an expression level of p55gamma gene or a p55gamma protein.

In some embodiments, the drug is a vector for gene therapy including a vector for gene editing, gene expression regulation, or gene delivery; alternatively, the drug is a vectorr for cell therapy including a stem cell, an immune cell, or a repair cell.

In some embodiments, a dosage form of the drug includes an oral liquid, an injection, a tablet, a pill, a dispersion, a capsule, a dropping pill, a granule, a suspension, and an emulsion.

The present disclosure has following beneficial effects:

The use of the p55gamma protein or the encoding gene thereof as the target in the preparation of the drug for preventing and/or treating the vascular calcification is proposed for the first time, and the p55gamma protein affects the vascular calcification by regulating phosphorylation of Yes-associated protein (YAP). It is proposed for the first time that the vascular calcification can be prevented and/or treated by overexpressing p55gamma gene or increasing the activity of the p55gamma protein.

### BRIEF DESCRIPTION OF THE DRAWINGS

FIG. 1A-FIG. 1H show the results of down-regulation of p55gamma during the vascular calcification, where FIG. 1A shows the mRNA level of p55gamma during calcification of mouse smooth muscle cells; FIG. 1B shows the protein expression result of p55gamma during calcification of mouse smooth muscle cells; FIG. 1C shows the mRNA level of p55gamma during calcification of human smooth muscle cells; FIG. 1D shows the protein expression result of p55gamma during calcification of human smooth muscle cells; FIG. 1E shows the mRNA level of p55gamma in the vascular calcification mouse model; FIG. 1F shows the protein expression result of p55gamma in the vascular calcification mouse model; FIG. 1G shows the mRNA level of p55gamma in the in vitro vascular ring calcification model; and FIG. 1H shows the immunofluorescence result in the vascular calcification mouse model;

FIG. 2A-FIG. 2L show the results of p55gamma overexpression in reducing vascular calcification, where FIG. 2A shows the expression result of p55gamma in the blood vessels of transgenic mice; FIG. 2B shows the results of staining vascular calcification in different groups of mice; FIG. 2C-FIG. 2I show the results of calcium ion content in tissues and blood vessels in different groups; FIG. 2J-FIG. 2K show the results of mRNA levels of calcification-related proteins RUNX2 and ALP in different groups; and FIG. 2L shows the protein level results of calcification-related protein ALP in different groups;

FIG. 3A-FIG. 3G show the results of p55gamma overexpression at the cellular level in reducing smooth muscle cell calcification, where FIG. 3A shows the p55gamma expression results in different groups; FIG. 3B shows the result of alizarin red staining; FIG. 3C shows the ALP activity of cells after stimulation with calcification medium for 2 days; FIG. 3D shows the calcium ion concentration; FIG. 3E shows the mRNA levels of calcification-related proteins RUNX2, BMP4, and ALP; and FIG. 3F-FIG. 3G show the protein levels of calcification-related proteins RUNX2, BMP4, ALP, and OPN;

FIG. 4A-FIG. 4L show the results of inhibiting p55gamma to aggravate vascular calcification, where FIG. 4A shows the expression of p55gamma in blood vessels of mice in different groups; FIG. 4B shows the staining results of mice in different groups; FIG. 4C-FIG. 4I show the calcium ion content in different tissues of mice in different groups; and FIG. 4J-FIG. 4L show the mRNA level of vascular calcification-related proteins in mice in different groups;

FIG. 5A-FIG. 5H show the results of low expression of p55gamma at the cellular level to aggravate cell calcification, where FIG. 5A shows the protein level of p55gamma in cells in different groups; FIG. 5B is the staining results showing calcium ion concentrations in smooth muscle cells in different groups; FIG. 5C shows the ALP activity in different groups; FIG. 5D shows the statistical result of calcium ion concentration in smooth muscle cells in different groups; and FIG. 5E-FIG. 5H show the mRNA levels of calcification-related proteins (RUNX2, BMP2, BMP4, and ALP) in different groups; and

FIG. 6A-FIG. 6H shows the results of high expression of p55gamma to alleviate vascular calcification by promoting YAP nuclear export, where FIG. 6A-FIG. 6B show the phosphorylation level of YAP1 after calcification culture; FIG. 6C shows the phosphorylation level of YAP1 after overexpression of p55gamma and calcification culture; FIG. 6D-FIG. 6E show the immunofluorescence result; and FIG. 6F-FIG. 6H show the detection of YAP1 content in different components of cells by nucleocytoplasmic separation.

### DETAILED DESCRIPTION OF THE EMBODIMENTS

The present disclosure provides use of a p55gamma protein or an encoding gene thereof as a target in the preparation of a drug for preventing and/or treating vascular calcification.

In the present disclosure, the p55gamma protein affects the vascular calcification preferably by regulating phosphorylation of a YAP. The effect is achieved by overexpressing p55gamma gene or increasing an activity of the p55gamma protein. A process of overexpressing p55gamma gene preferably includes adenovirus transfection; and the adenovirus transfection preferably include the following steps: transforming a p55gamma-overexpressed plasmid pAd-track-p55gamma into an Escherichia coli strain BJ5183 to allow recombination, selecting resulting single clones to allow identification by restriction analysis, transforming a resulting successfully-identified single clone into an Escherichia coli strain DH5A to allow bacterial solution amplification and plasmid extraction, and linearizing a resulting plasmid with a restriction endonuclease PAC1; transfecting a resulting linearized plasmid into a 293A cell to produce a recombinant adenovirus particle, and then infecting cells with the recombinant adenovirus particle or injecting the recombinant adenovirus particle into body to achieve the process of overexpressing p55gamma gene. There are no special restrictions on the specific sources of overexpression plasmid vector, plasmid carrying adenovirus genome, and 293 cell, and conventional commercially available products in the field can be used. There is no special limitation on the specific method of the transfection.

The present disclosure further provides a method for studying a function of genes related to vascular calcification, or screening a drug for preventing or treating the vascular calcification, or constructing a disease model of the vascular calcification, or analyzing a gene therapy drug of the vascular calcification, including overexpressing p55gamma gene or increasing an activity of the p55gamma protein.

In the present disclosure, the method is used to prevent or treat the vascular calcification in a cell in vitro, in an animal model in vivo, or in drug screening. A process of overexpressing p55gamma gene includes adenovirus transfection, and steps of the adenovirus transfection are the same as above and will not be repeated here.

The present disclosure further provides a drug for preventing and/or treating vascular calcification, where the drug is capable of increasing an expression level of p55gamma gene or a p55gamma protein.

In the present disclosure, the drug is a vector for gene therapy including a vector for gene editing, gene expression regulation, or gene delivery; alternatively, the drug is a vector for cell therapy including a stem cell, an immune cell, or a repair cell. A dosage form of the drug is preferably selected from the group consisting of an oral liquid, an injection, a tablet, a pill, a dispersion, a capsule, a dropping pill, a granule, a suspension, and an emulsion. The drug preferably further includes a pharmaceutical auxiliary material; there is no special limitation on specific types of the pharmaceutical auxiliary material, which depends on the specific pharmaceutical dosage form.

The technical solution provided by the present disclosure will be described in detail below with reference to the examples, but they should not be construed as limiting the claimed scope of the present disclosure.

In the following examples, all methods are conventional methods, unless otherwise specified.

All materials and reagents used in the following examples may be commercially available, unless otherwise specified.

### Example 1

Downregulation of p55gamma During Vascular Calcification

The expression of p55gamma in vascular calcification was detected in an in vitro cell calcification model and at the overall level, respectively.

At the cellular level, vascular smooth muscle cells were treated with calcium chloride and β-glycerophosphate, causing the smooth muscle cells to undergo osteogenic phenotype transformation.

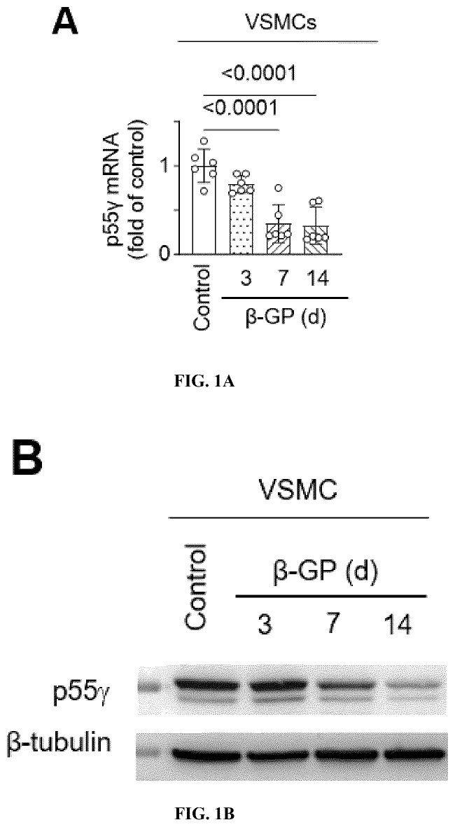

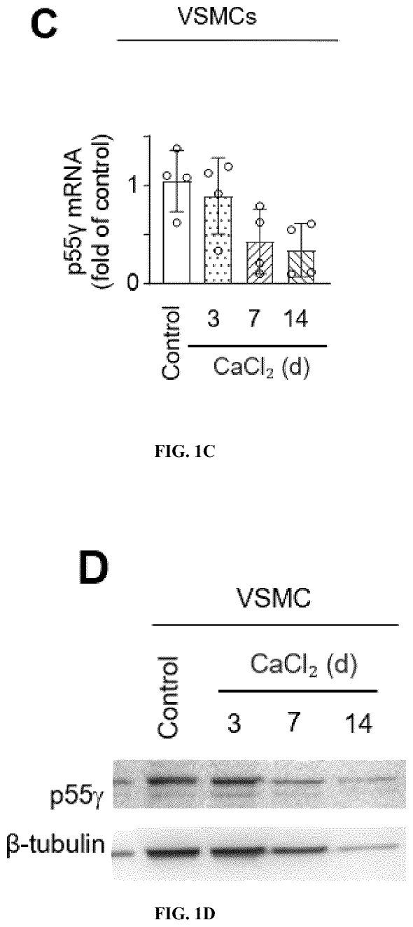

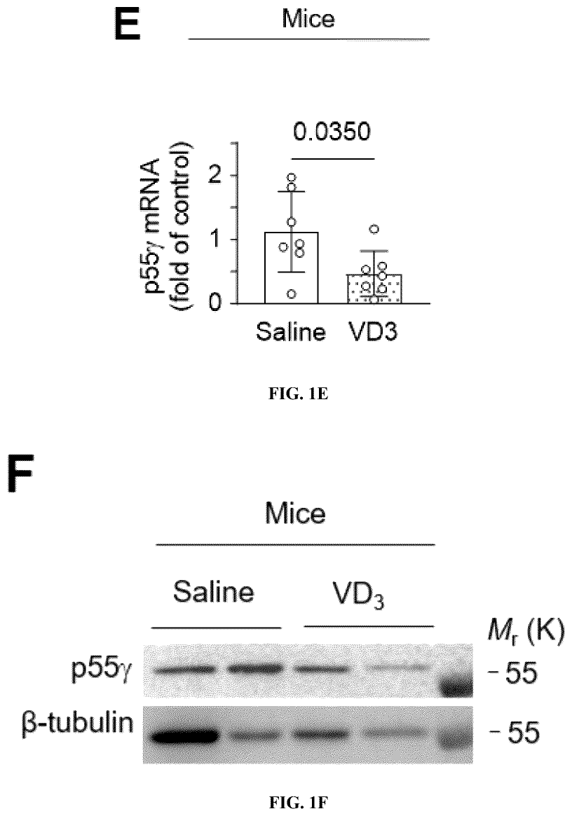

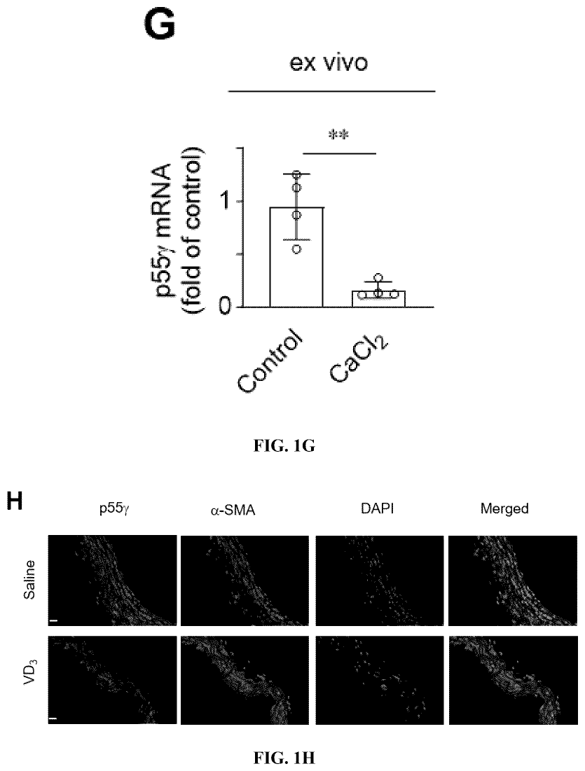

A final concentration of 2 mM calcium chloride and 10 mM β-glycerol phosphate were added into the mouse smooth muscle cell medium to stimulate for different times (0 d, 3 d, 7 d, and 14 d), and then cells were collected. RNA and protein were extracted, and mRNA level of p55gamma was detected by QPCR and the protein expression of p55gamma was detected by Western Blot. The results showed that the mRNA and protein levels of p55gamma were down-regulated during the calcification of mouse smooth muscle cells (FIG. 1A, FIG. 1B). The expression of p55gamma during calcification of human smooth muscle cells (calcification method was the same as above) was further detected, and the results showed that the mRNA and protein levels of p55gamma were also down-regulated (FIG. 1C, FIG. 1D).

Then the mouse model of vascular calcification was established. Wild-type C57 mice aged 12-16 weeks were injected subcutaneously with vitamin D₃ for 3 consecutive days and then recorded as VD3 group, while the mice of control group were injected with the same dose of physiological saline and then recorded as Saline group. On the 7th day, blood and thoracic aorta were collected from each group, and serum was used for subsequent calcium ion detection. After the entire blood vessel was fixed, alizarin red staining was conducted to evaluate whether calcification occurred in the blood vessel. A microCT scan was performed on the entire blood vessel to observe whether there was calcium salt deposition on the blood vessel. At the same time, the ascending main part of the blood vessel was taken, embedded in paraffin and sectioned, stained with Von Kossa and observed under a microscope to see if there was calcium salt deposition. Meanwhile, the tissues prone to calcification were taken, such as the heart, liver, lungs, and kidneys, the blood vessels and the above tissues were homogenized, the resulting supernatant was collected, and the calcium ion content in the blood vessels and other tissues was detected with a calcium ion detection kit. The expression changes of p55gamma were detected during the formation of vascular calcification. The thoracic aorta of mice was removed at different time points (3 d, 7 d). The thoracic aorta was quickly frozen in liquid nitrogen, and a first part of which was used to extract RNAs for QPCR to detect the mRNA expression of p55gamma gene; a second part of which was used to extract proteins for Western Blot to detect the expression of p55gamma protein. A third part of the thoracic aorta was make aortic sections after fixation for immunofluorescence staining to detect the expression of p55gamma protein. The results showed that the mRNA and protein levels of p55gamma were down-regulated during vascular calcification in mice (FIG. 1E, FIG. 1F). Immunofluorescence results showed that the fluorescence expression of p55gamma was down-regulated in calcified blood vessels (FIG. 1H).

Furthermore, an in vitro vascular ring calcification model was established. The blood vessels isolated from the mice were cut into 1 mm to 2 mm vascular rings, placed in DMEM medium with 10% FBS, 2 mM calcium chloride and 10 mM β-glycerol phosphate were added into the medium, and samples were collected 3 days later and RNA was extracted to detect the mRNA level of p55gamma gene. The results showed that the mRNA level of p55gamma gene was down-regulated during the vascular ring calcification in vitro (FIG. 1G).

### Example 2

p55gamma Overexpression in Reducing Vascular Calcification

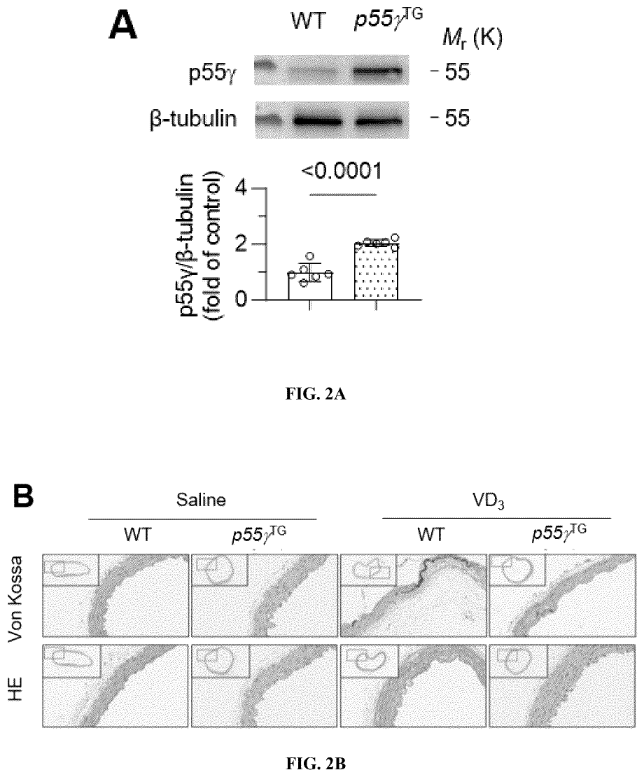

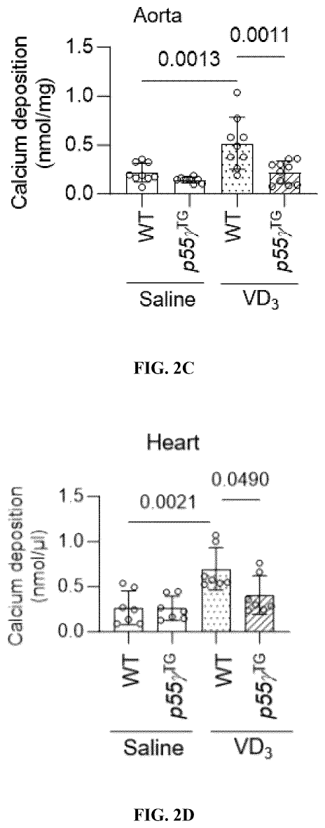

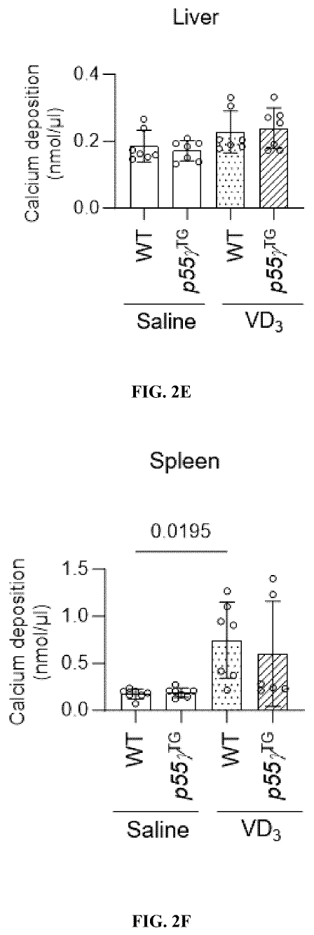

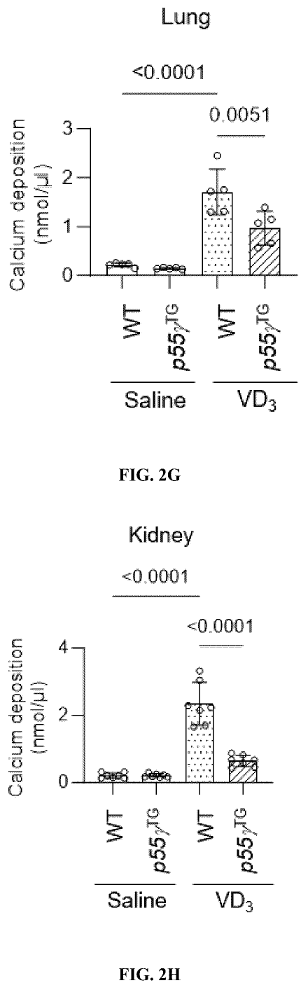

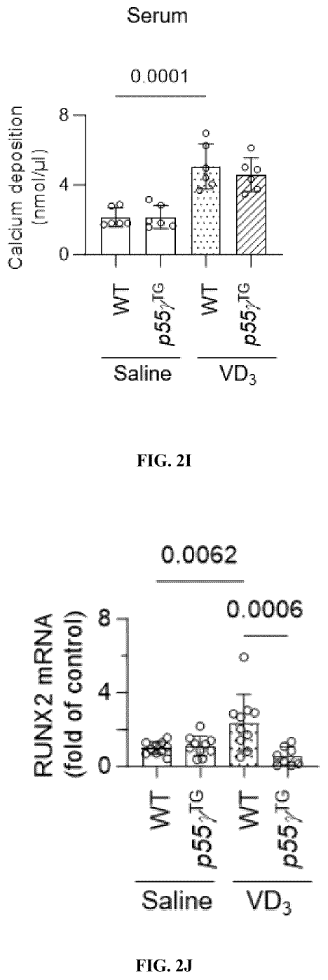

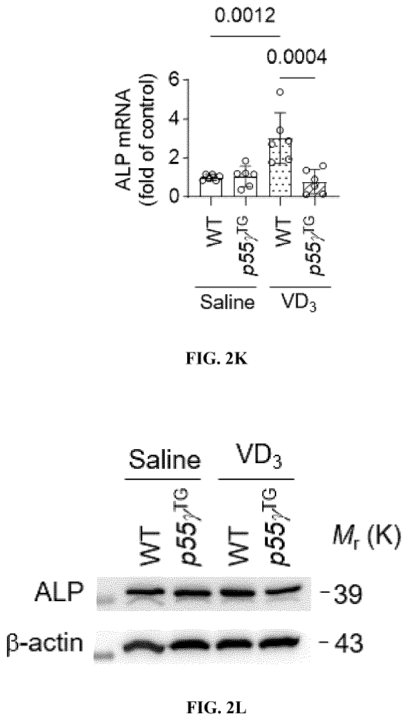

p55gamma transgenic mice were constructed. An exogenous gene p55gamma was constructed into the transposon plasmid piggyBac, which was microinjected into mouse fertilized eggs together with transposase mRNA under a microscope. The transposase excised an exogenous DNA fragment from the plasmid, inserted into the mouse genome, and then the fertilized eggs were injected into the oviduct of a female mouse. Postnatal mice were bred and genotyped to obtain p55gamm-overexpressed mice. The expression level of p55gamma in blood vessels of non-transgenic mice (WT) and p55gamma transgenic mice (p55yγ^{TG}) was detected, and FIG. 2A showed that p55gamma was highly expressed in blood vessels of transgenic mice.

p55gamma-overexpressed mice and littermate controls aged 12-16 weeks were selected and injected subcutaneously with vitamin D₃ or physiological saline. Transgenic mice injected subcutaneously with vitamin D₃ were recorded as the p55γ^{TG} group under VD₃; transgenic mice injected subcutaneously with physiological saline were recorded as the p55γ^{TG} group under Saline; littermate control wild-type mice subcutaneously injected with vitamin D₃ were recorded as the WT group under VD₃; littermate control wild-type mice subcutaneously injected with physiological saline were recorded as the WT group under Saline. All the mice were injected continuously for 3 d, and were sacrificed on the 7th day to collect blood and thoracic aorta, and the serum was used for subsequent detection of calcium ions. After the entire blood vessel was fixed, alizarin red staining was conducted to evaluate whether there were differences in vascular calcification. At the same time, the ascending main part of the blood vessel was taken, embedded in paraffin and sectioned, stained with Von Kossa and observed under the microscope to see whether there was any difference in calcium salt deposition. The results showed that vascular calcification was reduced in p55gamma transgenic mice (FIG. 2B). The tissues prone to calcification were taken, such as the heart, liver, lungs, and kidneys, the blood vessels and the above tissues were homogenized, the supernatant was collected, and the calcium ion content in the blood vessels was detected with the calcium ion detection kit. FIG. 2C-FIG. 2I showed that after calcification induction in p55gamma-overexpressed mice, the calcium ion contents in blood vessels, heart, lungs, and kidneys were reduced. On the 5th day after vitamin D₃ injection, the aorta was removed and snap-frozen in liquid nitrogen for subsequent RNA extraction or protein extraction, and the mRNA and protein levels of vascular calcification-related proteins were detected. The results showed that the mRNA levels of calcification-related proteins RUNX2 and ALP were reduced (FIG. 2J and FIG. 2K) and the protein levels of ALP were reduced (FIG. 2L).

### Example 3

p55gamma Overexpression in Attenuating Smooth Muscle Cell Calcification at the Cellular Level

At the cellular level, p55gamma adenovirus was used for high expression to construct the p55gamma overexpression plasmid. The primers were designed, including forward: 5′-atgtacaatacggtg tggag-3′ (SEQ ID NO: 1), reverse: 5′-ttatctgcaaagcgagggcatct-3′ (SEQ ID NO: 2). After the p55gamma fragment was amplified, the fragment was inserted into pAd-track to obtain the pAd-track-p55gamma plasmid. The p55gamma-overexpressed plasmid pAd-track-p55gamma was transformed into an Escherichia coli strain BJ5183 to allow recombination, resulting single clones were selected to allow identification by restriction analysis, the resulting successfully-identified single clone was transformed into an Escherichia coli strain DH5A to allow bacterial solution amplification and plasmid extraction, and the resulting plasmid was linearized with the restriction endonuclease PAC1; the resulting linearized plasmid was transfected into the 293A cell to produce the recombinant adenovirus particle, to increase the level of p55gamma in smooth muscle cells and study its effect on cell calcification.

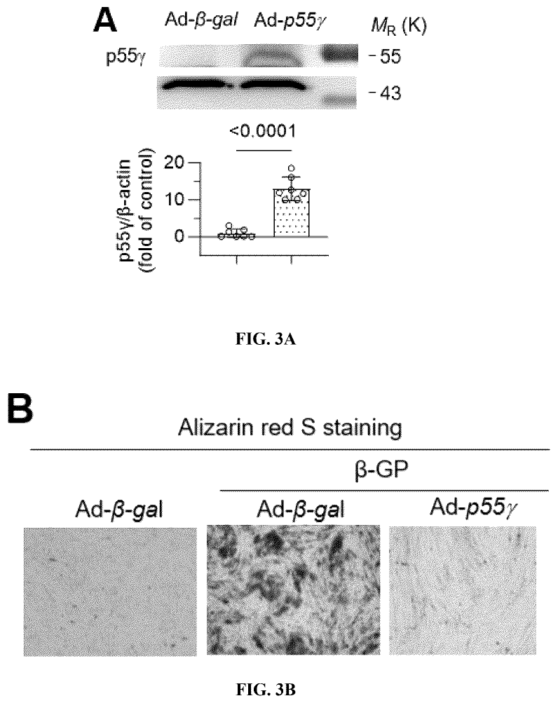

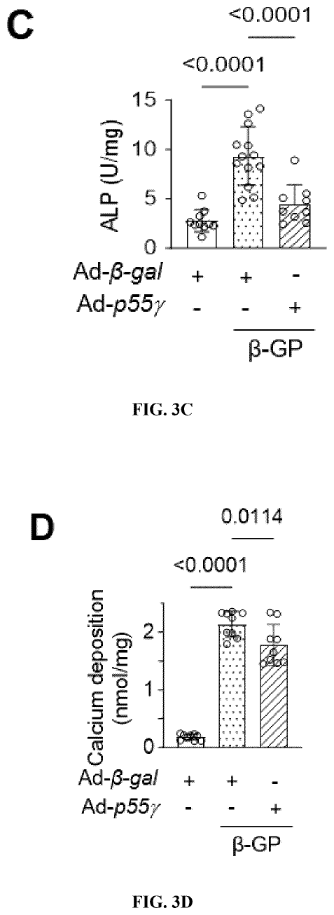

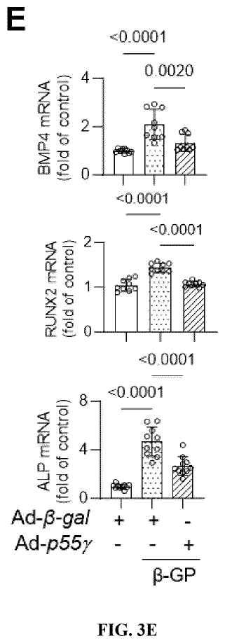

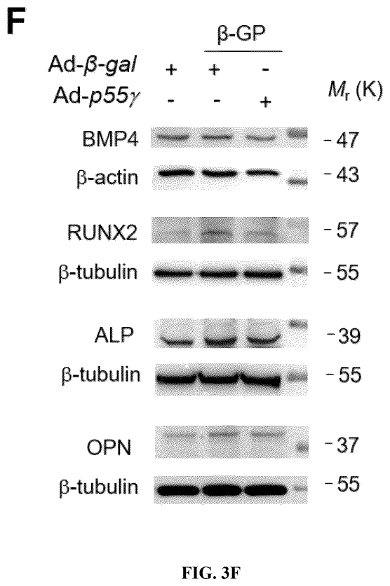

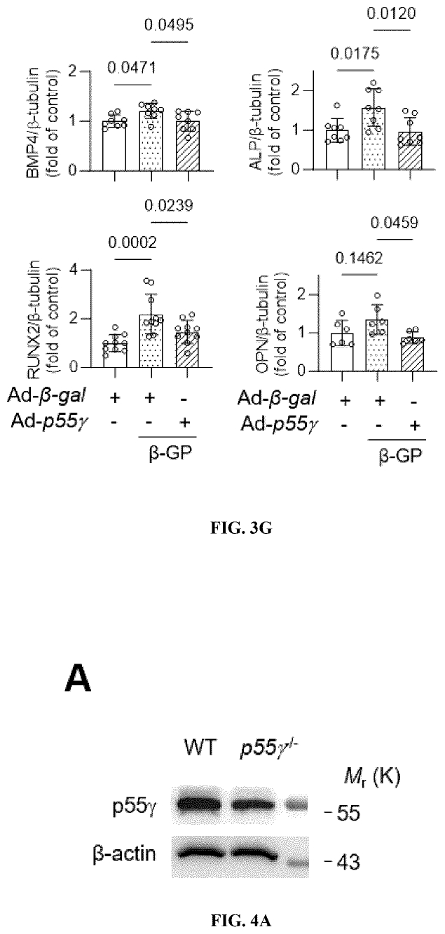

p55gamma adenovirus was added to the smooth muscle cells cultured in vitro, where the group with p55gamma adenovirus was recorded as the Ad-p55γ group, and the group without p55gamma adenovirus was recorded as the Ad-β-gal group. After 48 h, the cells were collected to extract protein and detect the protein level of p55gamma. The results showed that p55gamma was highly expressed in the group adding p55gamma adenovirus (FIG. 3A). After 14 d of stimulation with calcification medium containing 2 mM calcium chloride and 10 mM β-glycerol phosphate, those stimulated by calcification medium were recorded as the β-GP group. After fixation with PFA, they were stained with alizarin red and photographed for analysis. The results showed that high expression of p55gamma reduced the calcium ion concentration in smooth muscle cells (FIG. 3B, FIG. 3D). Cells were collected after stimulation with calcification medium for 2 d. The activity of ALP was detected, FIG. 3C showed that high expression of p55gamma reduced ALP activity. RNA and protein were extracted for QPCR to detect the mRNA levels of calcification-related proteins (RUNX2, BMP4, and ALP). The results showed that high expression of p55gamma inhibited the mRNA levels of RUNX2, BMP4, and ALP (FIG. 3E). Western blot results showed that high expression of p55gamma inhibited the protein levels of calcification-related proteins (FIG. 3F and FIL. 3G).

### Example 4

Inhibition of p55gamma in Aggravating Vascular Calcification

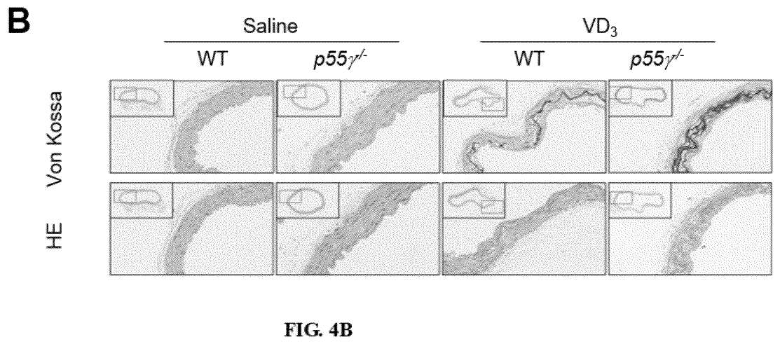

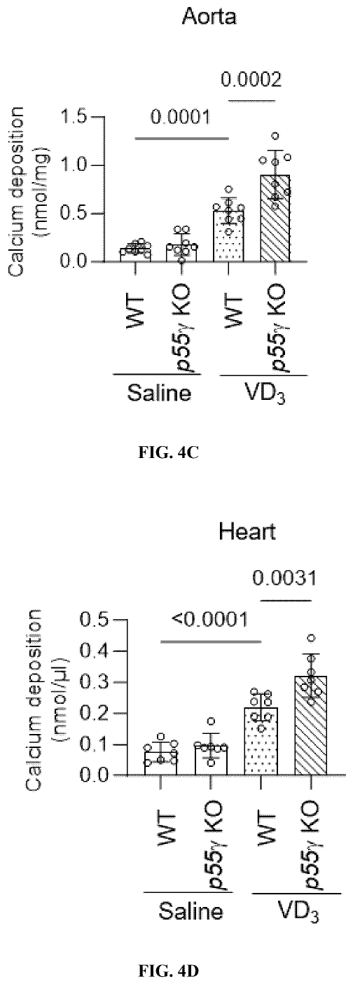

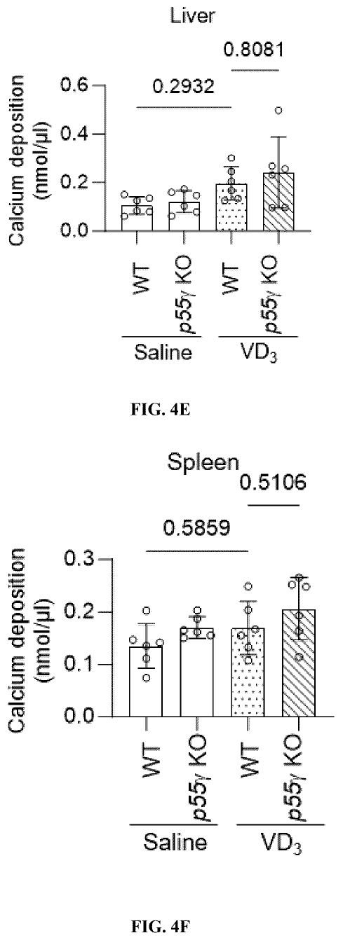

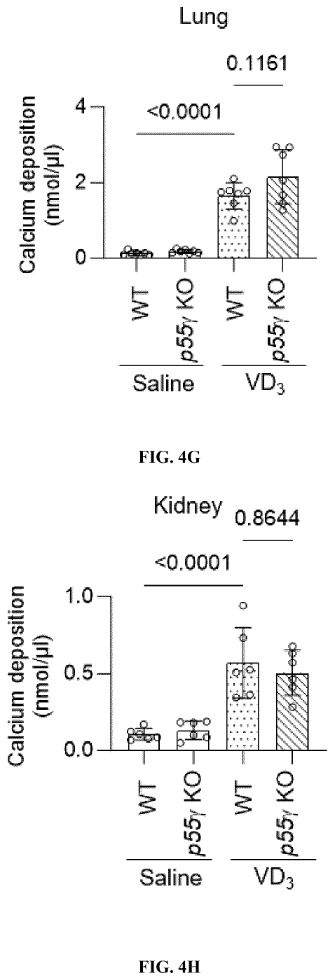

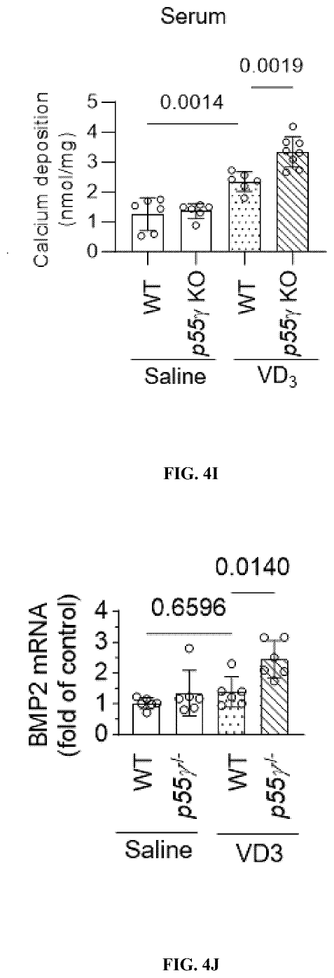

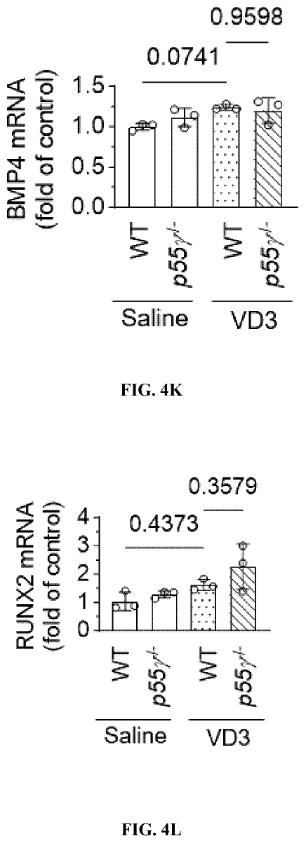

p55gamma smooth muscle cell-specific knockout mice were constructed. Two lop sites were inserted at both ends of the 3rd exon of p55gamma gene through homologous recombination, and resulting p55gamma-lop mice were crossed with tagln-cre mice to obtain the p55gamma smooth muscle cell-specific knockout mice (recorded as p55γ^{l-}), while mice not knocked out were recorded as the WT group. The expression level of p55gamma in blood vessels was detected. FIG. 4A showed that in the p55gamma smooth muscle cell-specific knockout mice, p55gamma was lowly expressed in mouse blood vessels.

p55gamma smooth muscle cell-specific knockout mice and littermate controls aged 12-16 weeks were selected and injected subcutaneously with vitamin D₃ or physiological saline. The p55gamma smooth muscle cell-specific knockout mice subcutaneously injected with vitamin D₃ were recorded as the p55γ^{l-} group under VD₃; the p55gamma smooth muscle cell-specific knockout mice injected subcutaneously with physiological saline were recorded as the p55γ^{l-} group under Saline; littermate control mice not knocked out and subcutaneously injected with vitamin D₃ were recorded as the WT group under VD₃; littermate control mice not knocked out and subcutaneously injected with physiological saline were recorded as the WT group under Saline. All the mice were injected continuously for 3 d, and were sacrificed on the 7th day to collect blood and thoracic aorta, and the serum was used for subsequent detection of calcium ions. After the entire blood vessel was fixed, alizarin red staining was conducted to evaluate whether there were differences in vascular calcification. At the same time, the ascending main part of the blood vessel was taken, embedded in paraffin and sectioned, stained with Von Kossa and observed under a microscope to see whether there was any difference in calcium salt deposition. The results showed that p55gamma smooth muscle cell-specific knockout mice had increased vascular calcification (FIG. 4B). The tissues prone to calcification were taken, such as the heart, liver, lungs, and kidneys, the blood vessels and the above tissues were homogenized, the supernatant was collected, and the calcium ion content in the blood vessels was detected with the calcium ion detection kit. FIG. 4C-FIG. 4I showed that p55gamma smooth muscle cell-specific knockout mice had increased calcium ion levels in blood vessels, heart, and serum after calcification induction. On the 5th day after vitamin D₃ injection, the aorta was removed and snap-frozen in liquid nitrogen for subsequent RNA extraction, and the mRNA level of vascular calcification-related proteins was detected. The results showed (FIG. 4J, FIG. 4K and FIG. 41) that the mRNA levels of calcification-related proteins BMP2 and RUNX2 were increased, while there was no significant difference in the mRNA levels of BMP4.

### Example 5

Low Expression of p55gamma at the Cellular Level in Aggravating Cell Calcification

At the cellular level, siRNA was used to knock down p55gamma expression and inhibit p55gamma levels in smooth muscle cells to study its effect on cell calcification.

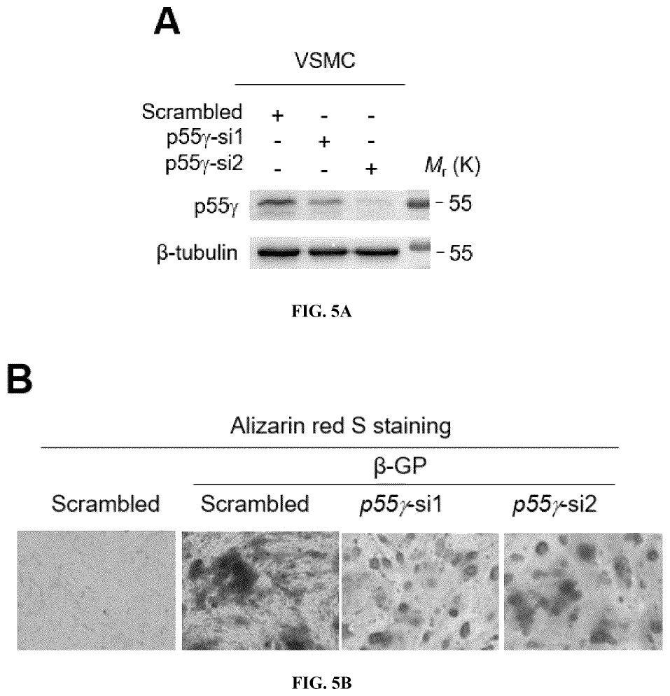

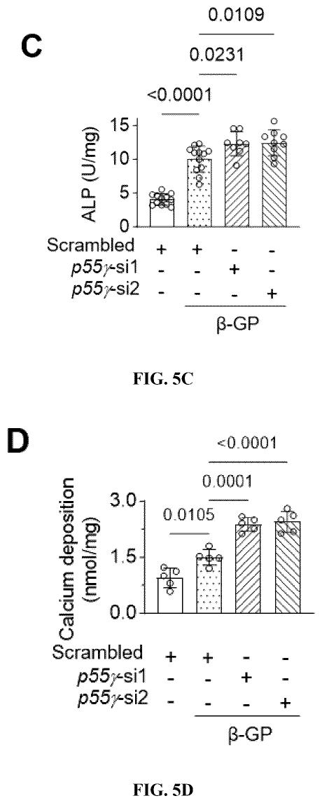

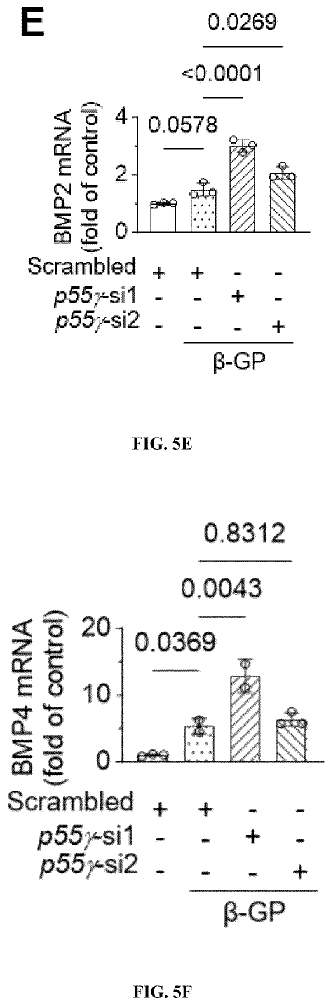

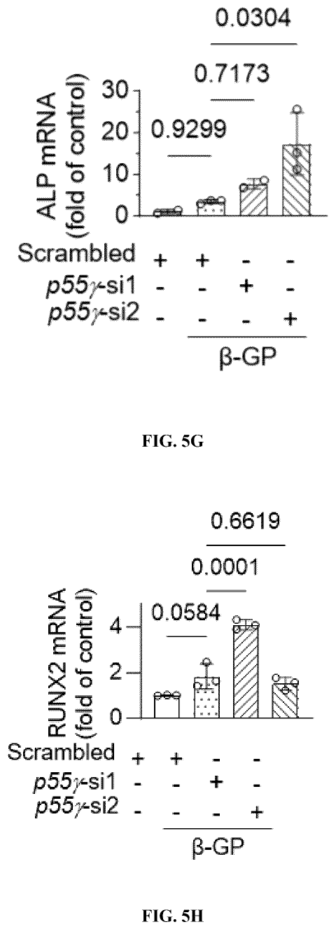

p55gamma siRNA (Si1: sense strand 5′-GCUCAGUACAAUCCCAAACUU-3′ (SEQ ID NO: 3), antisense strand: 5′-AAGUUUGGGAUUGUACUGAGC-3′ (SEQ ID NO: 4); SI2: sense strand 5′-GCUUUGGACAACCGAGAAAUA-3′ (SEQ ID NO: 5), antisense strand 5′-UAUUUCUCGGUUGUCCAAAGC-3′ (SEQ ID NO: 6)) was added to smooth muscle cells cultured in vitro. After 48 h, the cells were collected to extract protein and detect the protein level of p55gamma. The results showed that p55gamma was lowly expressed in the group adding siRNA (FIG. 5A). After 14 d of stimulation with calcification medium containing 2 mM calcium chloride and 10 mM β-glycerol phosphate, the cells were fixated with PFA and stained with alizarin red. The group that did not add siRNA and did not undergo calcification culture was recorded as the Scrambled group, the group that did not add siRNA but underwent calcification culture was recorded as the Scrambled group under β-GP, the group that added Si1 siRNA and underwent calcification culture was recorded as the p55γ-si1 group under β-GP, and the group that added Si2 siRNA and underwent calcification culture was recorded as the p55γ-si2 group under β-GP. Photographic analysis results showed that low expression of p55gamma increased calcium ion concentration in smooth muscle cells (FIG. 5B, FIG. 5D). Cells were collected after stimulation with calcification medium for 2 d. The activity of ALP was detected, FIG. 5C showed that low expression of p55gamma increased ALP activity. RNA and protein were extracted for QPCR to detect the mRNA levels of calcification-related proteins (RUNX2, BMP2, BMP4, and ALP). The results showed that low expression of p55gamma increased the mRNA levels of RUNX2, BMP2, BMP4, and ALP (FIG. 5E-FIG. 5H).

### Example 6

High Expression of p55gamma in Reducing Vascular Calcification by Promoting YAP Export from the Nucleus

In order to explore the mechanism of p55gamma regulating vascular calcification, adenovirus was used to highly express p55gamma at the cellular level (the specific method was the same as in Example 3). Samples were collected 3 d after calcification induction, and RNA was extracted for high-throughput sequencing to search for the downstream target genes of p55gamma.

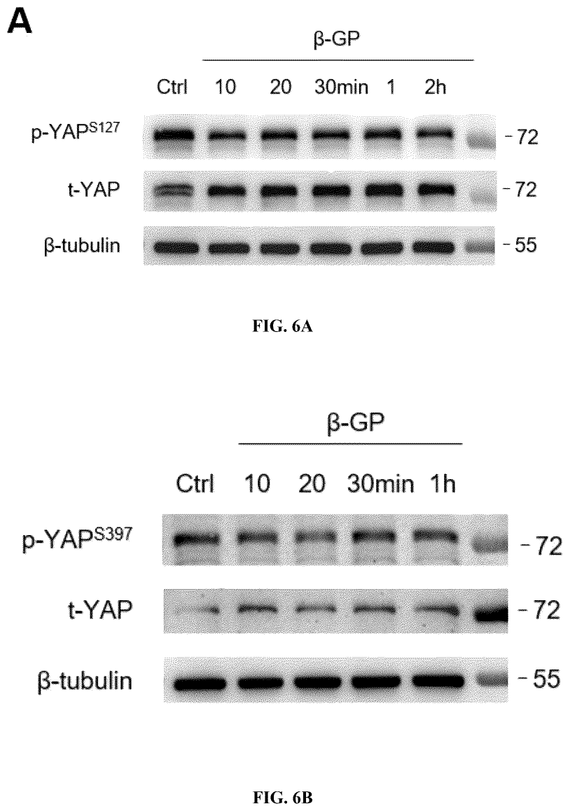

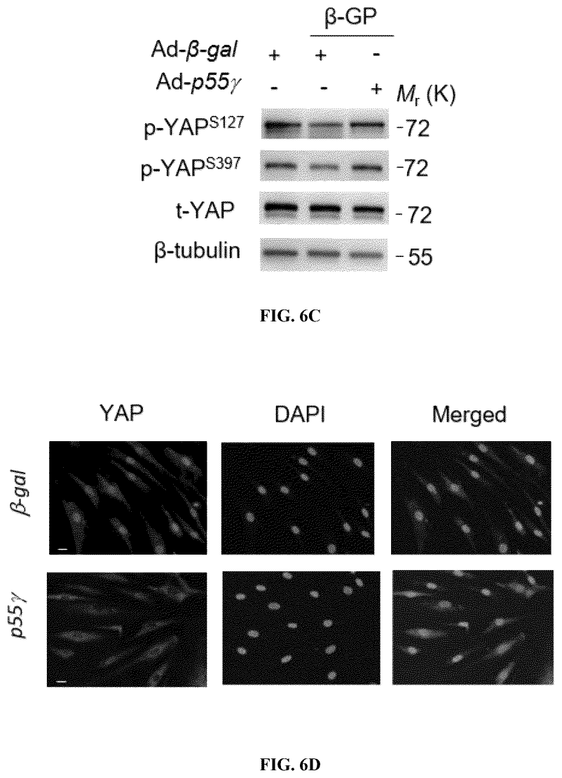

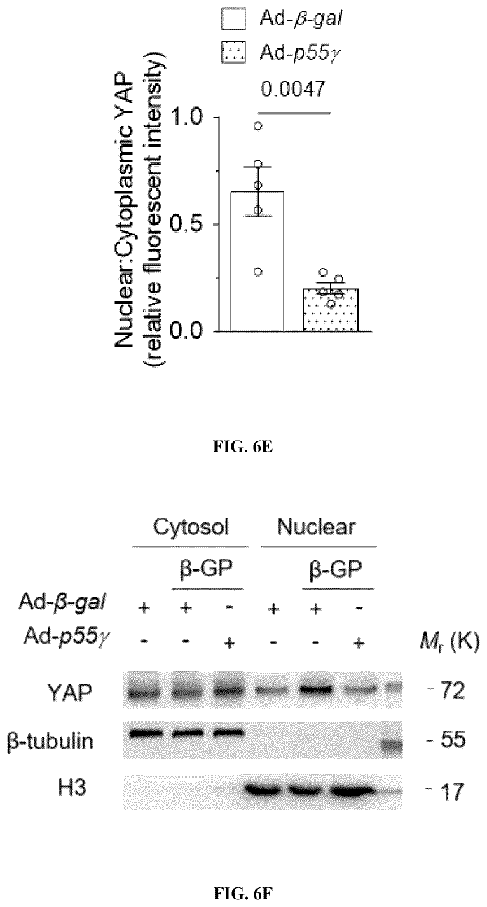

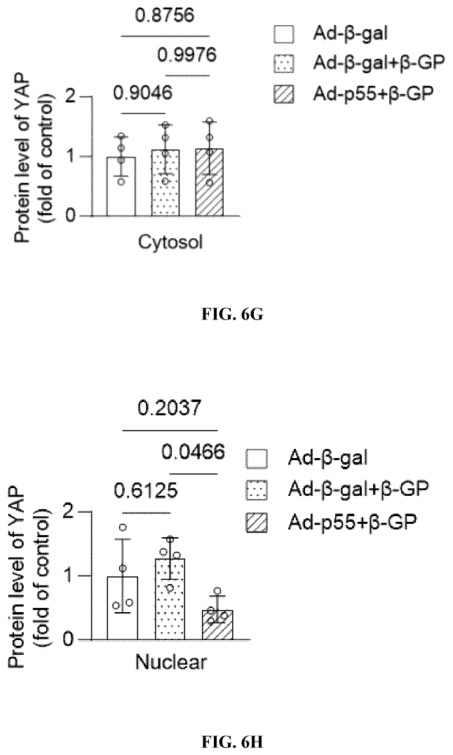

The differential gene pathway enrichment results showed that the Hippo signaling pathway was significantly enriched. Compared with the control group, p55gamma overexpression inhibited the Hippo signaling pathway. GSEA analysis results showed that p55gamma overexpression reduced the expression level of YAP1. The phosphorylation levels of YAP1 at positions 127 and 397 regulated the entry of YAP1 into the nucleus and affected the transcription and translation of downstream target genes. Therefore, changes in the phosphorylation level of YAP1 during calcification were first detected. The calcification medium was added into smooth muscle cells, and samples were collected at 10 min, 20 min, 30 min, 1 h, and 2 h to extract proteins and detect the phosphorylation of YAP1. The results showed that the phosphorylation level of YAP1 decreased when calcification medium was added for 10 min, 20 min, and 30 min (FIG. 6A and FIG. 6B). p55gamma was overexpressed in smooth muscle cells, and samples were collected to detect the phosphorylation level of YAP1 10 min after calcification induction. The results showed that high expression of p55gamma increased the phosphorylation level of YAP1 (FIG. 6C) and promoted YAP1 export from the nucleus. Immunofluorescence results showed that the YAP content in the nucleus decreased after high expression of p55gamma (FIG. 6D and FIG. 6E). Furthermore, the content of YAP1 in different components of cells was detected through nucleocytoplasmic separation. The results showed that high expression of p55gamma did not affect the YAP1 content in the cytoplasm, but the YAP1 content in the nucleus was reduced (FIG. 6F, FIG. 6G and FIG. 6H). Based on the above results, p55gamma promoted the phosphorylation of YAP1, causing YAP1 to exit the nucleus, thereby inhibiting the expression of downstream calcification-related genes and inhibiting vascular calcification.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preventing and/or treating vascular calcification, comprising administering a pharmaceutical composition comprising a p55gamma protein or an encoding gene thereof as a target to a subject in need thereof.

2. The method according to claim 1, wherein the p55gamma protein affects the vascular calcification by regulating phosphorylation of a Yes-associated protein (YAP).

3. The method according to claim 1, wherein preventing and/or treating vascular calcification is achieved by overexpressing p55gamma gene or increasing an activity of the p55gamma protein.

4. The method according to claim 3, wherein a process of overexpressing p55gamma gene comprises adenovirus transfection; and the adenovirus transfection specifically comprises the following steps: transforming a p55gamma-overexpressed plasmid pAd-track-p55gamma into an Escherichia coli strain BJ5183 to allow recombination, selecting resulting single clones to allow identification by restriction analysis, transforming a resulting successfully-identified single clone into an Escherichia coli strain DH5A to allow bacterial solution amplification and plasmid extraction, and linearizing a resulting plasmid with a restriction endonuclease PAC1; transfecting a resulting linearized plasmid into a 293A cell to produce a recombinant adenovirus particle, and then infecting cells with the recombinant adenovirus particle or injecting the recombinant adenovirus particle into body to achieve the process of overexpressing p55gamma gene.

5. A method for studying a function of genes related to vascular calcification, or screening a drug for preventing or treating the vascular calcification, or constructing a disease model of the vascular calcification, or analyzing a gene therapy drug of the vascular calcification, comprising overexpressing p55gamma gene or increasing an activity of a p55gamma protein.

6. The method according to claim 5, wherein the method is used to prevent or treat the vascular calcification in a cell in vitro, in an animal model in vivo, or in drug screening.

7. The method according to claim 5, wherein a process of overexpressing p55gamma gene comprises adenovirus transfection; and the adenovirus transfection specifically comprises the following steps: transforming a p55gamma-overexpressed plasmid pAd-track-p55gamma into an Escherichia coli strain BJ5183 to allow recombination, selecting resulting single clones to allow identification by restriction analysis, transforming a resulting successfully-identified single clone into an Escherichia coli strain DH5A to allow bacterial solution amplification and plasmid extraction, and linearizing a resulting plasmid with a restriction endonuclease PAC1; transfecting a resulting linearized plasmid into a 293A cell to produce a recombinant adenovirus particle, and then infecting cells with the recombinant adenovirus particle or injecting the recombinant adenovirus particle into body to achieve the process of overexpressing p55gamma gene.

8. A drug for preventing and/or treating vascular calcification, wherein the drug is capable of increasing an expression level of p55gamma gene or a p55gamma protein.

9. The drug according to claim 8, wherein the drug is a vector for a gene therapy comprising a vector for gene editing, gene expression regulation, or gene delivery; alternatively, the drug is a vector for a cell therapy comprising a stem cell, an immune cell, or a repair cell.

10. The drug according to claim 8, wherein a dosage form of the drug is selected from the group consisting of an oral liquid, an injection, a tablet, a pill, a dispersion, a capsule, a dropping pill, a granule, a suspension, and an emulsion.


SEQUENCE LISTING (6 sequences)

SEQ ID NO: 1 - DNA, 20 bp, synthetic construct
  source 1..20: mol_type = other DNA; note = forward primer for constructing the p55gamma overexpression plasmid
atgtacaatacggtgtggag

SEQ ID NO: 2 - DNA, 23 bp, synthetic construct
  source 1..23: mol_type = other DNA; note = reverse primer for constructing the p55gamma overexpression plasmid
ttatctgcaaagcgagggcatct

SEQ ID NO: 3 - RNA, 21 nt, synthetic construct
  source 1..21: mol_type = other RNA; note = sense strand of Si1
gctcagtacaatcccaaactt

SEQ ID NO: 4 - RNA, 21 nt, synthetic construct
  source 1..21: mol_type = other RNA; note = antisense strand of Si1
aagtttgggattgtactgagc

SEQ ID NO: 5 - RNA, 21 nt, synthetic construct
  source 1..21: mol_type = other RNA; note = sense strand of SI2
gctttggacaaccgagaaata

SEQ ID NO: 6 - RNA, 21 nt, synthetic construct
  source 1..21: mol_type = other RNA; note = antisense strand of SI2
tatttctcggttgtccaaagc